(19) United States
(12) Patent Application Publication
(10) Pub. No.: US 2026/0144654 A1
(43) Pub. Date: May 28, 2026

(54) BRIM FOR PROSTHETIC SOCKET

(71) Applicant: Ossur Iceland ehf, Reykjavik (IS)

(72) Inventors: Margret Sol RAGNARSDOTTIR, Reykjavik (IS); Oskar Thor LARUSSON, Reykjavik (IS); Franziska Simone BARZ, Reykjavik (IS); Hogna HRINGSDOTTIR, Reykjavik (IS); Stefan Orn STEFANSSON, Reykjavik (IS); Andri ORRASON, Reykjavik (IS)

(21) Appl. No.: 19/399,031

(22) Filed: Nov. 24, 2025

Related U.S. Application Data
(60) Provisional application No. 63/724,570, filed on Nov. 25, 2024.
(60) Provisional application No. 63/876,313, filed on Sep. 5, 2025.

(51) Int. Cl. A61F 2/78

(52) U.S. Cl. CPC A61F 2/7812; A61F 2002/7825

(57) ABSTRACT

A brim, method, and prosthetic socket system includes a brim configured to secure onto the proximal end of a prosthetic socket. The brim has a cuff section extending proximally from the outer edge of the socket and an interface section that forms a pocket with at least one lip that extends over the socket's proximal end. The brim may incorporate reinforcement or textile layers to improve adhesion with a resin-impregnated socket blank and may include flexible strap regions that create a flexion brim, allowing for limb movement. The method involves placing fiber braids and textile layers over a mandrel, inserting the proximal fiber ends into the brim pocket, and applying resin to create a continuous structure. The brim or finished socket assembly provides enhanced durability, flexibility, and comfort, while eliminating the need for post-grinding of the proximal socket edge and offering a seamless cosmetic finish.

### FIELD OF THE DISCLOSURE

The disclosure relates to prosthetic devices and, more particularly, to an improved brim attachable to a proximal end of a prosthetic socket.

### BACKGROUND

A typical prosthetic leg and foot include a socket, a pylon, and a foot. The socket serves as the prosthetic component that encases and conforms to the residual limb or stump, to which prosthetic elements such as the foot are attached. The socket must fit snugly around the residual limb to ensure a secure connection and adequate support, while also maintaining sufficient looseness to promote proper circulation. When appropriately fitted, the socket effectively transfers loads from the residual limb to the ground.

The prosthetic socket is the interface where the residual limb or stump of the amputated limb meets the prosthetic limb, establishing the connection between human tissue and the prosthetic limb. It provides a surface for weight-bearing, offers additional support during ambulation, and contributes to the overall control of the prosthetic system. An ill-fitting socket may result in complications such as persistent pain, sores, or blisters on the residual limb. The socket may be manufactured to be preferably rigid and capable of being sealed or, once donned, converted into a hypobaric chamber, incorporating a valve mechanism to expel air. The socket may be alternatively semi-rigid.

A prosthetic liner serves as an interface between the socket and the residual limb, enhancing comfort during use. It comprises an elastomeric body with a closed distal end and an open proximal end that is pulled or rolled over the residual limb. The liner adheres to the residual limb and serves as a transitional interface between the limb and the rigid components of the socket, facilitating a smoother transition of contact.

A brim may be incorporated into the prosthetic socket and can offer greater flexibility than the socket itself. The brim may serve as a transition from the rigidity of the prosthetic socket to the residual limb, especially at the peripheral edge of the socket. It may also enhance comfort and mitigate the occurrence of blisters, pain, and sores on the soft tissue of the residual limb. Furthermore, the softness and resilience of the brim—particularly when contrasted with the inherent rigidity of the prosthetic socket—may accommodate volume fluctuations and minor movements of the residual limb at the interface between the socket and the limb, thereby providing an intermediate level of support and connection.

The brim may be configured to be placed in tensioned and relaxed positions, thereby assisting the donning and continual wear of the socket (i.e., tensioned in use, relaxed for donning and doffing). The brim is preferably configured to attach or secure about an upper or proximal perimeter of the socket and extend over, beyond, or secure to the inner and outer surfaces of the socket.

Prosthetic sockets have been formed using various techniques, including plaster-of-Paris molds, computer modeling, vacuum forming, and other methods commonly used by prosthetists. A socket may be made according to U.S. Pat. No. 5,885,509, granted Mar. 23, 1999; U.S. Pat. No. 5,971,729, granted Oct. 26, 1999; and U.S. Pat. No. 7,105,122, granted Sep. 12, 2006, each of which is incorporated herein by reference.

Examples of a brim are found in U.S. Pat. No. 9,474,635, granted Oct. 25, 2016; U.S. patent application publication no. 2024/0252331 A1, published on Aug. 1, 2024, each of which is incorporated herein by reference. A drawback of known brims is their tendency to tear due to a mismatch between the brim's flexibility and the socket's rigidity, particularly at the interface between the proximal peripheral edge of the socket and the brim.

The brim is desired to soften the edge of the socket to prevent ripping of the liner, a prosthetic sleeve, or clothing. Moreover, in the example of a transtibial amputee, it is desired to provide medial and lateral stability while offering support and comfort to the amputee's residual limb.

Based on the above observations, it is desirable to develop a brim that is more durable than existing ones while maintaining its soft and flexible characteristics. Additionally, a brim that adheres better to a rigid or semi-rigid prosthetic socket is sought, due to the differences in material properties and types between the socket and the brim.

### SUMMARY

According to various embodiments, a brim is designed to attach to a prosthetic socket and to move between a tensioned and a relaxed position during donning and doffing. The brim is intended to secure around the upper or proximal edge of the socket and to extend beyond or attach to the inner and outer surfaces of the socket. [0014] The cuff section forms the upper part of the brim and extends upward from the socket opening. The cuff section can shift between a tensioned and a relaxed state. It is flexible relative to the socket, permitting distension along an axis of the socket so that the cuff section can bend relative to the socket.

The interface section defines the lower portion of the brim and extends along the inner surface of the socket. For example, the interface section may be configured to extend flush against the inner surface of the socket, and the socket may incorporate a recess that extends into its thickness relative to regions outside the recess. The interface section may be positioned within the recess and against the inner surface of the socket, in conjunction with the areas of the inner surface outside the recess, thereby forming a continuous inner surface without interruption.

A challenge associated with silicone materials, which are preferred for the fundamental structure of the brim, is that they may pose difficulties in adhesion using an adhesive. Historically, it has been observed that brims tend to detach from the socket; therefore, the brim is engineered to attach firmly to the socket during repeated cycles of donning and doffing.

In the tensioned position, the cuff section may be arranged to fold downward away from the opening and over the outer surface of the socket, thereby facilitating donning and doffing. The cuff and interface sections may be fabricated integrally from a single material, thus providing a more integrated or cohesive, and durable brim.

The brim may exhibit varying thicknesses to accommodate different degrees of flexibility and durability around its circumference. The proximal end may be constructed with greater thickness and may taper either uniformly or variably towards the distal end. The interface section may have a consistent, uniform height around the brim's circumference, or it may vary, as exemplified by the upper section, which features an asymmetrical height that originates at the transition section.

In a particular variation, a sleeve with a brim at its distal end may be provided and arranged modularly for either permanent or temporary attachment to a socket.

Numerous additional advantages, features, and functions of various embodiments of a brim will become readily apparent and better understood through the following description and the accompanying drawings.

### BRIEF DESCRIPTION OF THE DRAWINGS

FIG. 1 is a perspective view of a prior art prosthetic socket and brim attached.

FIG. 2A is a first-perspective view of a brim embodiment, showing the anterior aspect of the brim on a prosthetic socket.

FIG. 2B is a second perspective view of the embodiment shown in FIG. 2A, showing the posterior aspect of the brim on a prosthetic socket.

FIG. 3A is a partial perspective view of an embodiment of the brim attached to a textile layer.

FIG. 3B is a perspective view of a reinforcement layer for the brim of FIG. 3A.

FIGS. 4A-4D illustrate perspective views of the positioning of the socket blank and one or more textile layers.

FIG. 4E is a perspective view of a cosmetic textile layer integrated with the brim of FIG. 3A.

FIG. 4F is a perspective view of the socket blank configured to receive the cosmetic textile layer of FIG. 4E.

FIG. 5A is a perspective view of a variation of the embodiment in FIG. 2A attached to a socket blank before resin impregnation.

FIG. 5B is a perspective view of the embodiment of FIG. 5A after resin impregnation of the socket blank.

FIG. 6 is a perspective view of the first embodiment of a socket having a flexion brim on the posterior aspect of the socket.

FIG. 7 is a perspective view of a second embodiment of a socket having a flexion brim on the posterior aspect of the socket.

FIG. 8 is a perspective view of a brim attachment with a flexion brim incorporated into the posterior aspect of the brim.

FIG. 9 is a perspective view of a socket assembly, including a fastener assembly for removably attaching a brim to a socket.

FIG. 10 is a perspective view of the casting assembly for forming a socket, including the fastener assembly, and being adapted to receive a brim.

FIG. 11 is a sectional view showing the detachment of the fastener assembly.

FIG. 12 is a perspective view of another embodiment of the brim.

FIG. 13 is an exemplary view of a brim with contours for different aspects of the proximal periphery, corresponding to a transtibial amputee.

FIG. 14A is a first exemplary view of another embodiment taken from an anterior perspective, illustrating a detachable sleeve having a brim section, for attachment to a socket.

FIG. 14B is another view of the embodiment shown in FIG. 14A, taken from a posterior perspective.

FIG. 14C illustrates a top perspective view of the embodiment of FIG. 14A showing an interior surface of the sleeve.

The drawing figures are not necessarily drawn to scale, but instead are drawn to provide a better understanding of the components thereof. They are not intended to limit the scope but to provide exemplary illustrations.

### GLOSSARY

The terms set forth below will have the meanings as defined.

The term “anterior” has its ordinary meaning and refers to a location ahead of or to the front of another. The term “posterior” also has an ordinary meaning and refers to a location behind or the rear of another location.

The term “distal” has its ordinary meaning and refers to a location farther from the heart than another location. The term “proximal” also has an ordinary meaning and refers to a location closer to the heart than another location.

The terms “rigid,” “flexible,” and “resilient” may distinguish characteristics of portions of certain features. The term “rigid” should denote that an element lacks flexibility. Within the context of “rigid features,” it should indicate that they do not lose their overall shape when force is applied and may break if bent with sufficient force. The term “flexible” should denote features capable of repeated bending, such that the features may be bent into retained shapes, or the features retain no general shape but continuously deform when force is applied. The term “resilient” should denote an element or feature that is not easily broken and may withstand continued use or movement.

The term “semi-rigid” means stiff and solid, but not inflexible.

The term “elastic” means that a structure can resume its normal shape spontaneously after contraction.

The term “textile” means a woven or knit fabric formed from interlocking fibers, filaments, or yarns. The term “fabric” may be made through weaving, knitting, spreading, felting, stitching, crocheting, or bonding that may be used in the production of further products.

The terms “integral” or “integrally knit” or “integral construction” mean that components are attached to, formed with, or joined to, and function as a single article in one piece. “Integral” by itself means being manufactured together simultaneously; that is, being made together as one part and not two separately manufactured parts. “Integral” also means at least one textile element that extends between different textile and/or sleeve areas.

The term “generally” is meant to connote “mostly” or “near exactly,” but not necessarily exactly, and refers to at least 90% as much.

The term “user” refers to a person who wears the socket. The user may be a patient or an operator. The term “clinician” refers to a clinical specialist, supervisor, therapist, doctor, or person with a similar role that assists or oversees the operation of the user's fabrication and observance of the socket.

The term “monolithic” denotes a unitary structure, which is not provided by a chemical or mechanical fusion of multiple structures but is formed from and defines a single mass of material consistently of the same type (for example, a structure formed by injection molding from an elastomer).

The term “anisotropic” denotes a physical property with a different value when measured in different directions.

The term “flexion brim” marks a region of the brim that is more flexible than other locations of the brim or, if the brim is omitted, at least one strap in favor of a solid structure. By use of the term “strap,” it is intended to mean a portion or additional feature of the brim extending across a recess, clearance, or other contoured periphery portion or geometry of the socket from side to side thereof. A strap may refer to a single strap or to a plurality of straps.

It will be understood that unless a term is defined to possess a described meaning, there is no intent to limit the meaning of such term, either expressly or indirectly, beyond its plain or ordinary meaning. The embodiments of the disclosure are adapted for a human body and may be dimensioned to accommodate different body sizes, shapes, and contours.

These and other features, aspects, and advantages of the present disclosure will be better understood in the following description, appended claims, and accompanying drawings.

### Detailed Description of Various Embodiments

### Prior Art

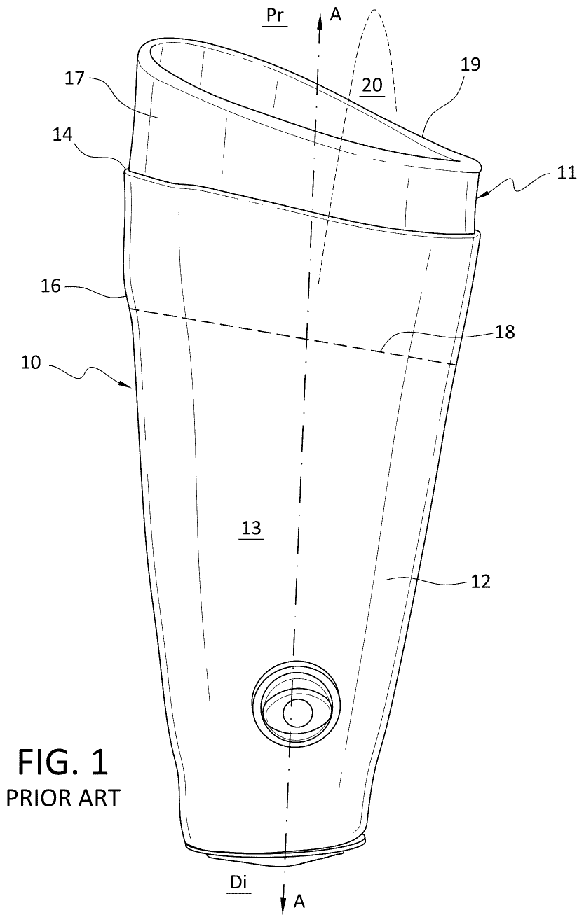

FIG. 1 illustrates a prior art brim 11 securing to a proximal end of a prosthetic socket 10, as found in U.S. patent application publication no. 2024/0252331 A1. The prosthetic socket 10 defines an opening 20 at the proximal end, Pr, and an outer surface 13. The brim 11 is coaxial to a central axis A-A of the socket and preferably extends circumferentially about the proximal end of the prosthetic socket 10. The brim 11 is tubular.

The brim 11 is coextensive with the outer surface 13 of the socket wall 12, such that the brim 11 projects from the prosthetic socket 10 along the same trajectory as the socket wall 12 extends in a proximal direction, Pr. A slight bulge 16 may be apparent along the outer surface 13 of the socket wall 12 as the prosthetic socket 10 accommodates the brim 11 and ensures the brim 11 is continuous with a contour or profile of the socket wall 12. As shown in FIG. 1, bulge 16 may correspond to a circumferential or peripheral distal brim edge, demarcating its termination at line 18. The brim 11 includes a cuff section 17 arranged to flexibly extend about and upwardly or proximally from a peripheral proximal edge 14 of the prosthetic socket 10.

An interface section (not shown) may define a lower or distal portion of the brim and extend distally from the cuff section 17. The interface section preferably adheres to the inner surface of the socket 10. The interface section may be coextensive with the inner surface of the socket 10, without interruption, along their combined continuous profile as a single trajectory and without any discernible break or seam to the user.

### Embodiments of the Disclosure

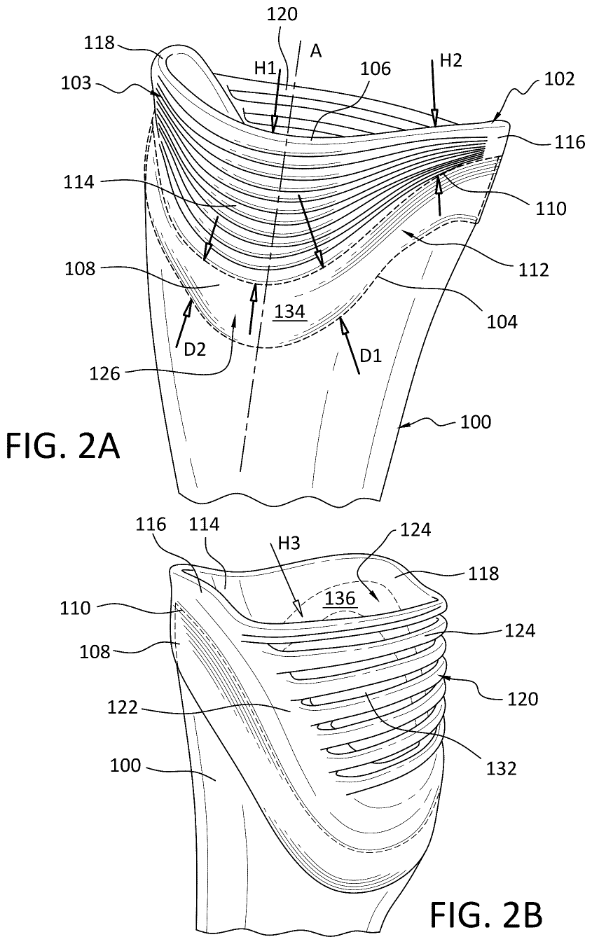

FIGS. 2A and 2B illustrate a brim 102 for securing to the proximal end or edge of a prosthetic socket 100. As in FIG. 1, the socket defines an opening at the proximal end and outer and inner surfaces. The brim 102 is coaxial to the socket's central axis.

The brim 102 includes a cuff section 103 arranged to flexibly extend about and upwardly or proximally from a peripheral proximal edge 110 of the socket and an interface section 112 defining a lower or distal portion of the brim extending distally from the cuff section 103. The interface section 112 forms a lip 134, 136 arranged to extend over a proximal end portion 108 of the prosthetic socket 100, including the peripheral proximal edge 110 of the socket, and secures therewith.

The interface section 112 forms a pocket 126, having at least a first lip 134 arranged to extend over an outer side of the proximal end portion 108 of the prosthetic socket 100. The brim 102 may include a second lip 136 arranged to extend over an inner side of the proximal end portion 108 of the prosthetic socket 100. The pocket 126 may include one or more lips on the outer or inner sides.

As discussed below, braids or fibers of the socket blank 128 (i.e., fiber braids with a distal attachment according to the Direct Socket method discussed below and provided herewith in an appendix) may be tucked in the pocket 126, such that when the socket blank 128 is injected with resin, the resin, socket blank 128 and at least one or more of the lips 134, 136 secure together. It will be noted that the fibers may require cutting to shape to enable being tucked between the lips before injecting the resin. Advantageously, the pocket 126, including at least one lip 134, 136, covers the peripheral proximal edge 110 of the socket, thereby eliminating the tedious process and work of grinding the peripheral proximal edge 110 of the socket after fabrication, which is a common practice in conventional prosthetic sockets, even with the prior art brim, mentioned above.

In another configuration, the brim 102 may be formed by additive manufacturing or other known molding or manufacturing techniques. As discussed above, braids or fibers of the socket blank 128 may be tucked in the pocket 126 of the brim 102, such that when the socket blank 128 is injected with resin, the resin, socket blank 128, and at least one or more of the lips, preferably both lips, secure together.

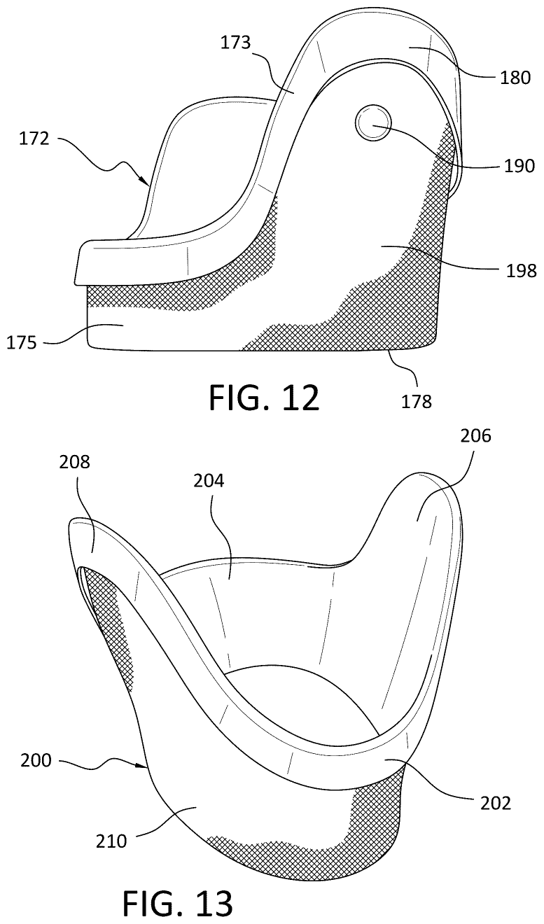

The basic structure of the brim encompassing the cuff section 103 and the interface section 112 may be formed by an elastomer, preferably silicone. Preferably, the cuff section 103 and the interface section 112 are formed and molded from a single silicone mass, thereby forming a monolithic, unitary, contiguous structure. When applicable, an outer fabric or fabric cover, as shown in FIG. 12, may be attached to the interface section 112 to improve the adherence of the resin of the prosthetic socket to the brim.

Examples of possible silicone formulations and general teachings for making a brim are found in at least U.S. Pat. No. 11,312,071, granted on Apr. 26, 2022, and incorporated herein by reference. Other suitable elastomeric materials may likewise be used.

In observing the configuration of the brim in FIGS. 2A and 2B, the brim 102 may be configured to a shape of the proximal periphery of the socket, particularly for a transtibial amputee, but not limited thereto. For example, a distal edge 104 of the brim 102 varies in distance D1, D2 from the peripheral proximal edge 110 of the socket about the opening at the proximal end to adapt to an anterior aspect of the residual limb. In another example, a distal edge 104 of the brim may be uniform, deviating no more than 5% from the peripheral proximal edge 110 of the socket about the opening at the proximal end. Likewise, a proximal edge 106 of the brim 102 may vary in height H1, H2 from the peripheral proximal edge 110 of the socket about the opening at the proximal end.

In the example of a transtibial amputee and thus prosthetic socket, as shown in the example of FIGS. 2A and 2B, an anterior portion 114 of the cuff section 103 may have a greater height H1 than a height H2 at a medial or a lateral portion 116, 118 of the cuff section 103. A posterior portion 120 of the cuff section 103 may have a greater height H3 than a height H2 at a medial or a lateral portion 116, 118 of the cuff section 103. The anterior portion 114 of the cuff section 103 may have a smaller height H1 than a height H3 at the posterior portion 120 of the cuff section 103. However, the brim may be configured to accommodate a user's anatomy and is not limited to the configurations shown herein.

In any of the embodiments described herein, the brim is adapted to fold over the socket to facilitate donning and doffing of the socket without damaging the prosthetic liner, clothing, or other objects. Accordingly, the thickness of the brim may vary depending upon its circumference, as taught in U.S. patent application publication no. 2024/0252331 A1, and likewise, be sufficiently thick to fully protect the user from the proximal peripheral edge of the prosthetic socket, particularly if there is no post-processing of the proximal peripheral edge (i.e., grinding to form a rounded edge).

FIG. 2B shows the posterior portion 120 of the cuff section 103, defining at least one strap 124 arranged laterally relative to the central axis A-A of the prosthetic socket 100 that forms a “flexion brim.” Specifically, the posterior portion 120 of the cuff section 103 defines at least two straps 124 spaced apart by a clearance 132. The posterior portion 120 of the cuff section 103 forms a frame section 122 extending outwardly relative to the peripheral proximal edge 110 of the socket. The strap 124 extends from and is confined within the frame section 122.

The strap 124 is preferably flexible, and flexibility can be achieved through the individual configuration of each strap, which may vary, and through the material composition of the brim. The strap 124 may be more flexible in the vertical or proximal direction to provide user comfort during knee bending, but more rigid in the medial or lateral directions to support the user's residual limb while walking. Likewise, the anterior, medial, or lateral portions of the brim may also include straps, either contiguous or non-contiguous with the straps on the posterior portion.

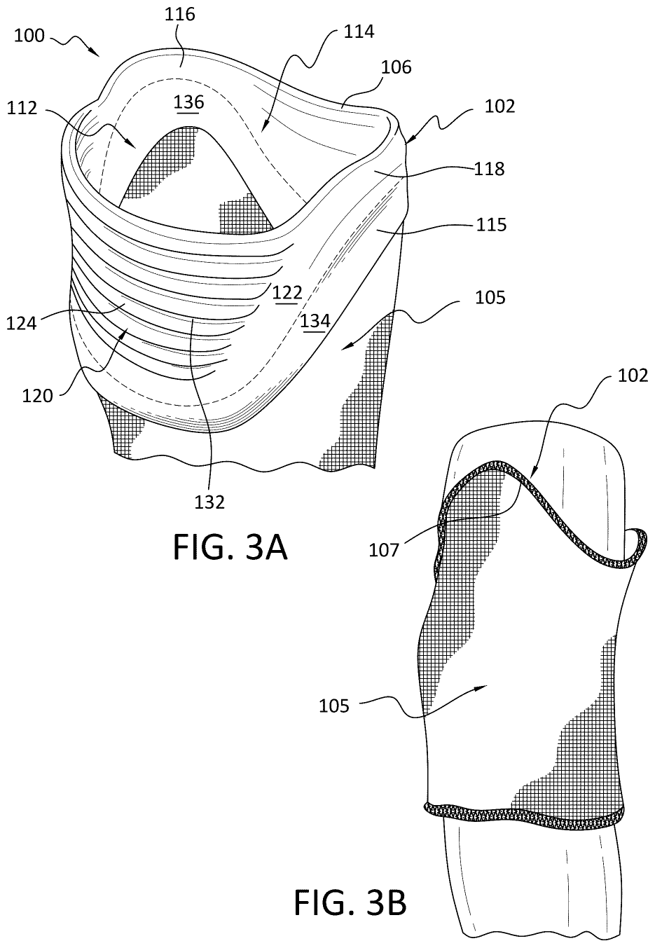

FIG. 3A shows a partial perspective view of an embodiment of the brim 102 attached to at least one textile layer 105. Integration of the brim 102 to at least one textile layer 105 can be like that of the integration of the brim 102 to the proximal end of a prosthetic socket 100, as discussed above. In other words, it is to be understood that the connections between the disclosed embodiments of the brim and socket likewise apply to connections between the brim and at least one textile layer. The cuff section 103 of the brim 102 is arranged to flexibly extend about and upwardly or proximally from a proximal end portion 115 of the at least one textile layer 105. The interface section 112, defining a lower or distal portion of the brim 102, extends distally from the cuff section 103. The interface section 112 forms a lip 134, 136, arranged to extend over the proximal end portion 115 of the at least one textile layer 105 and to secure therewith.

In an embodiment, the brim 102 can include a reinforcement layer 107 at the proximal end portion 115 of the at least one textile layer 105. FIG. 3B is a perspective view of the reinforcement layer 107 for the brim 102. The reinforcement layer 107 forms part of the brim 102 and advantageously reduces the number of fabrication steps when forming the brim 102 using additive manufacturing or other known molding or manufacturing techniques. The reinforcement layer 107 is arranged between at least one textile layer 105 and can be sewn to or integrated into at least one of the textile layers 105.

In an embodiment, the reinforcement layer 107 includes a fibrous element that is stitched between one or more textile layers 105, FIG. 3B illustrates the reinforcement layer 107 as a single, continuous reinforcement layer, wherein the brim 102 is additively manufactured or printed on top of the reinforcement layer 107. The reinforcement layer 107 can include one or more patches that are placed in the medial or a lateral portions 116, 118 or “ears” of the cuff section 103, wherein the patches of the reinforcement layer 107 do not reach all the way through (e.g., from proximal to distal ends of) the at least one textile layer 105. The reinforcement layer may be co-cured with the brim or subsequently laminated thereto.

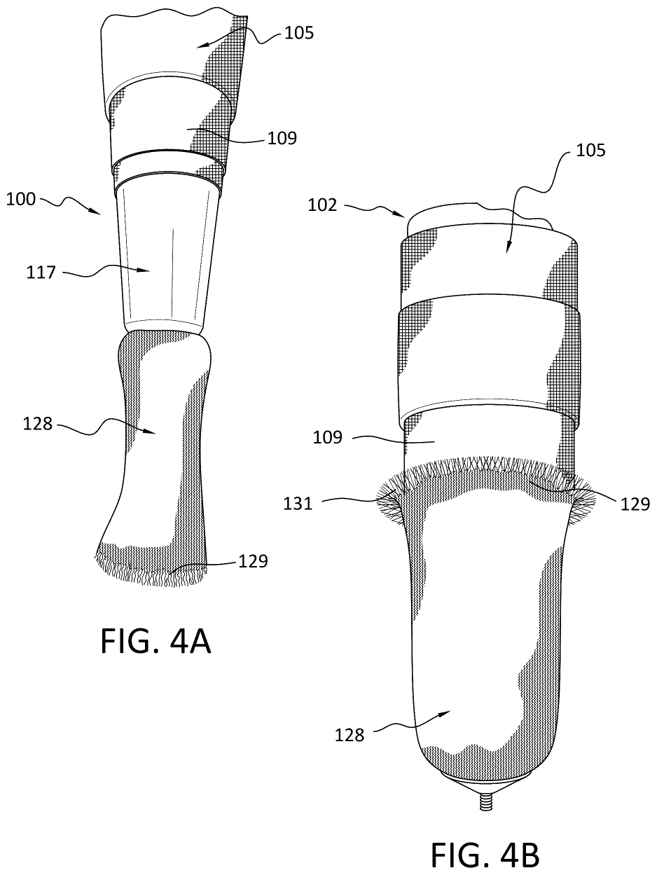

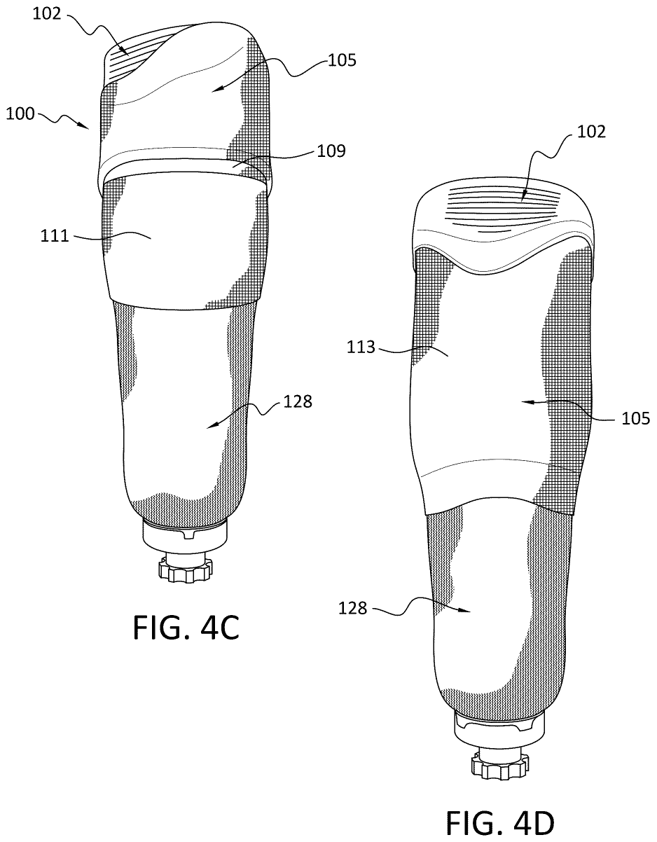

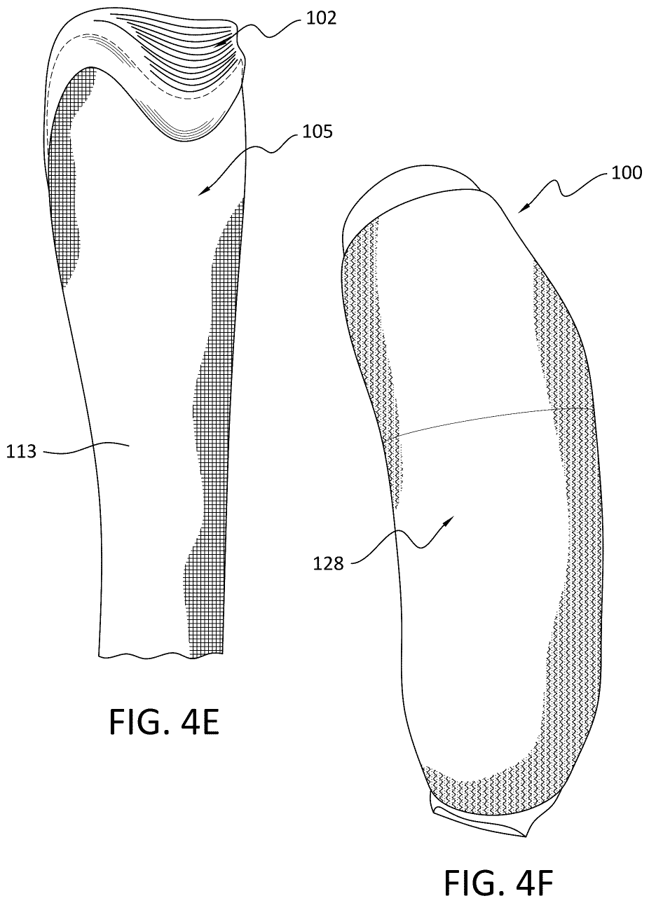

FIGS. 4A-4D illustrate perspective views of forming the socket 100 with multiple textile layers 105. As observed in FIG. 4A, the socket blank 128 is configured to integrate with one or more textile layers 105. The socket blank 128 is positioned to overlap with a first textile layer 109 (i.e., inner textile layer) of the one or more textile layers 105. The socket blank 128 can be assembled on a stump model 117 or mandrel to assemble the socket 100. As observed in FIG. 4B, the socket blank 128 overlaps the first textile layer 109 when assembled on the stump model 117. Fiber braids 129 of the socket blank 128 are cut and tapered to form a tapered portion 131 of the socket blank 128. The fiber braids 129 are cut below the brim 102 and tapered (i.e., cut at different lengths) so that the thickness of the socket blank 128 and one or more textile layers 105 is evened out. By providing the tapered portion 131 and allowing the fiber braids 129 to flare out and become detangled, a smoother transition is provided between the socket blank 128 and the one or more textile layers 105.

The embodiment depicted in FIGS. 4A-4D illustrate multiple textile layers 105, e.g., four textile layers, to thicken the top part of the brim 102 (i.e., the proximal portion of the socket 100). Since the fiber braids 129 are cut more distally from the brim 102, less material of the socket blank 128 is provided near the proximal portion of the socket 100. Cutting the fiber braids 129 lower facilitates easier fabrication of the socket 100, as otherwise the CPO would need to cut the fiber braids 129 more precisely to form to fit the socket blank 128 just below the brim 102. To compensate for the structural integrity of the socket 100 proximal to the tapered portion 131, multiple textile layers 105, and/or reinforcement layers 107, are provided to strengthen the proximal end of the socket 100.

For example, FIG. 4C shows a second textile layer 111, or intermediate layer, of the one or more textile layers 105 placed over the socket blank 128, including the tapered portion 131, and also placed over the first textile layer 109. In an embodiment, the second textile layer 111 can be distinct from the first textile layer 109 and configured to be placed on the socket blank 128 from the distal end upwards to hold fiber braids 129 in place. The second textile layer 111, in the form of a thin textile band, covers the fiber ends at the tapered portion 131 to keep the fiber braids 129 in place.

In FIG. 4D, a third textile layer 113, or outer layer, of the one or more textile layers 105 is arranged over the socket blank 128 and the second textile layer 111. The third textile layer 113 allows the cut fiber braids 129 to remain in place and facilitates easier fabrication of the socket using additive manufacturing. The third textile layer 113 reduces the profile of the tapered portion 131 and provides a smooth transition between the layers of the socket blank 128 and the one or more textile layers 105. Finally, a resin material can be applied to saturate one or more textile layers 105, fully integrating the brim 102 with the socket 100.

FIG. 4E is a perspective view of the third textile layer 113 as a cosmetic textile layer for arrangement over the socket of FIG. 4F. Because the one or more textile layers 105 can be noticeable on the outside of the socket 100 after fabrication (e.g., the one or more textile layers 105 being shorter than a full length of the socket 100 and therefore noticeable), the third textile layer 113 can be provided in length to covers all fiber braids 129 of the fiber blank 128, i.e., strength from the brim 102 to the distal end of the socket 100. Advantageously, the third textile layer 113 can be arranged to provide a consistent surface structure or smoothness, wherein lines between the brim 102 and the normal fiber-saturated socket are not visible. In other words, the third textile layer 113 can provide a consistent, aesthetically pleasing outer texture for the socket 100.

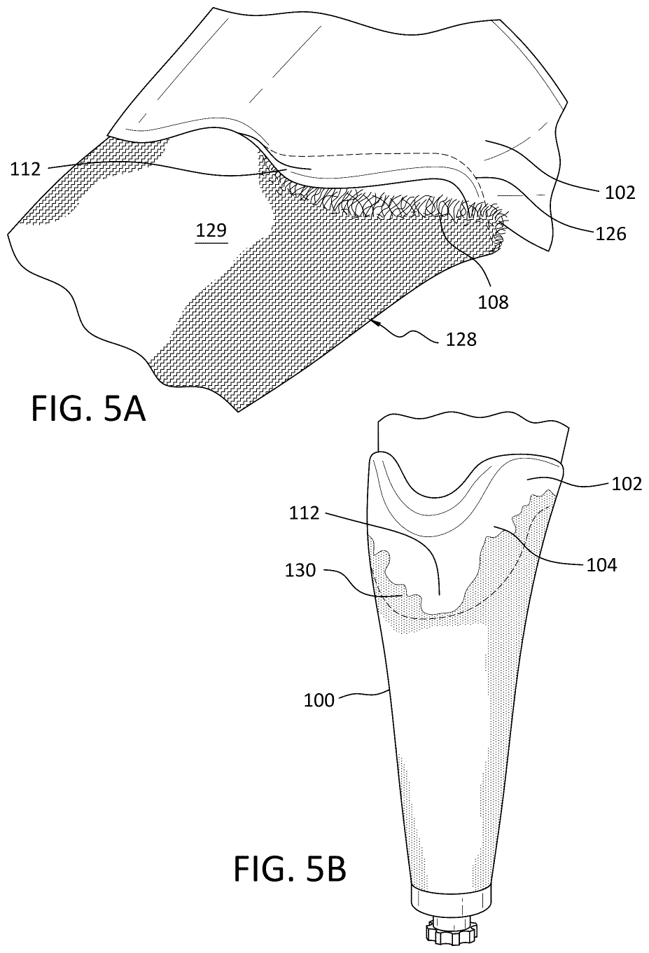

FIGS. 5A and 5B illustrate how the brim 102 can replace a brim or be included in a Direct Socket method, as taught in U.S. Pat. Nos. 5,885,509, 5,971,729, and 7,105,122, and with the “Instructions for Use Direct Socket TT” or “Instructions for Use Direct Socket TF’ provided by Össur hf, of Reykjavik, Iceland, copies of which are filed herewith in the appendix, and incorporated by reference.

The socket fabrication process is modified when the fiber braids 129 of the socket blank 128 are attached with a distal attachment. According to the revised method, the brim 102 is placed on the limb. The prosthetist places fibers on the residual limb but cuts them shorter to match the lip or lips, as in the lips 134, 136 discussed above, or pocket 126 on the brim 102. In an embodiment, the brim 102 has an interface 112 forming a pocket 126 into which the proximal end 130 of the arrangement of fiber braids 129 is inserted. The fibers are folded underneath the silicone lip on the brim 102. The prosthetist then follows the current process of injecting resin to form the socket, and the fibers allow the resin to flow beneath this lip.

When the socket is removed from the user, the need to grind down the proximal edge depends on the security of the brim and socket attachment. A secure attachment of the brim to the socket with this “lip” removes the tedious process and “dirty work” of grinding the socket after fabrication. A loose brim attachment to the socket with this “lip” may require the brim to be folded over.

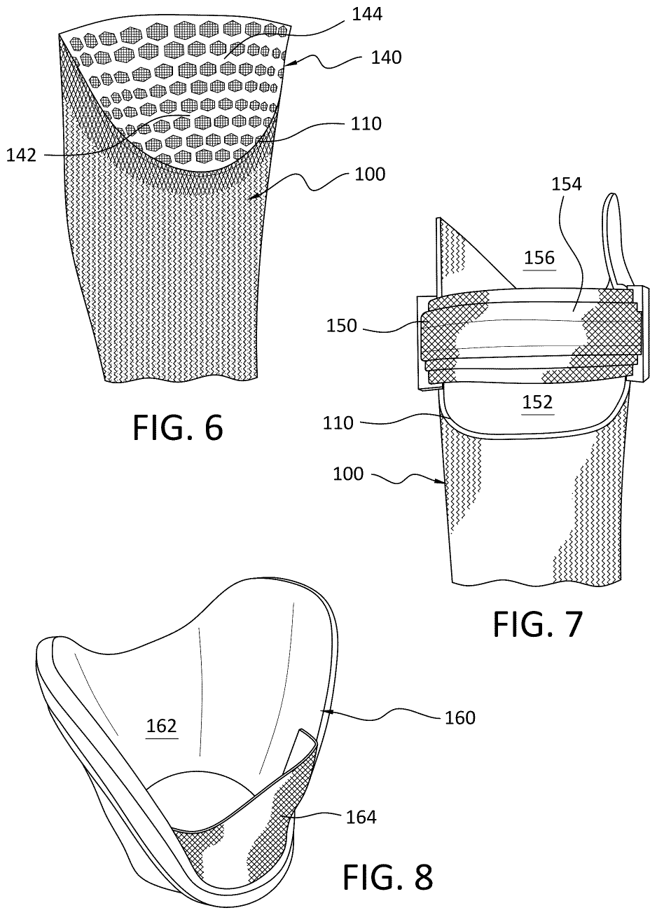

FIGS. 6-8 exemplify different flexion brims that may be used in different embodiments. In certain embodiments, the strap (124) forming the flexion brim may replace or supplement the posterior portion (120) of the cuff section shown in FIGS. 2A-2B.

In FIG. 6, the flexion brim 140, 150, 160 is for securing to a proximal end of a prosthetic socket 100, with each brim including a strap 144, 154, 164 arranged to extend across a proximal recess 142, 156 of the socket or 162 of the brim or across a distal recess 152 of the socket, and attached to opposed sides of the socket subtending the proximal recess. Each strap, 144, 154, and 164, may be a segment of flexible fabric. As shown, the proximal recess may be defined on the posterior aspect of the socket. However, the recess may be disposed of at other locations at the proximal end of the socket.

FIGS. 6 and 7 exemplify how the strap 144, 154 is provided with the socket without a brim, whereas FIG. 8 shows how the brim 160 defines a recess 162 and the strap 164 extends within at least part of the recess 162. FIG. 6 shows the strap 144 as distending the entirety of the recess 142, whereas FIG. 7 shows the strap 154 as extending across only a section of the recess 152, 156.

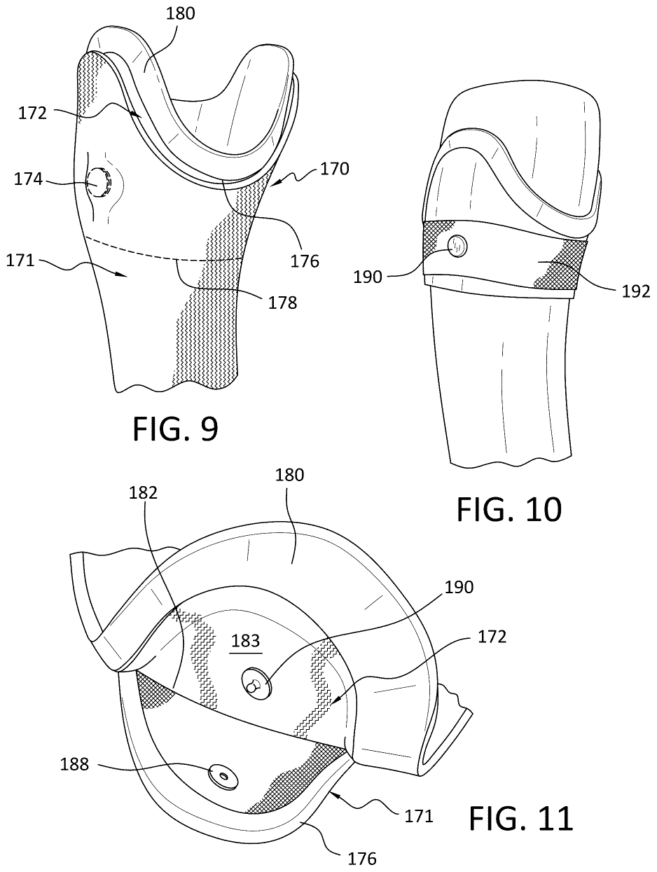

FIGS. 9-12 illustrate another embodiment of a socket assembly 170 with a prosthetic socket 171 and a brim 172, which is removable from the prosthetic socket 171. The prosthetic socket 171 is fabricated with a fastener assembly 174 that enables the brim 172 to be removed. As in other embodiments, the brim 172 is arranged to secure to a proximal end of a prosthetic socket 171. The socket defines an opening at the proximal end, as well as the outer and inner surfaces. The brim 172 is coaxial to the central axis of the prosthetic socket 171.

The brim 172 includes a cuff section 173 arranged to flexibly extend about and upwardly or proximally from a peripheral proximal edge 176 of the prosthetic socket 171. The brim 172 also includes an interface section 175 defining a lower or distal portion of the brim extending distally from the cuff section 173. The interface section 175 includes a fastener 190, arranged as part of a fastener assembly 174, for removably securing the fastener 190 to the prosthetic socket 171.

The socket assembly 170 includes the prosthetic socket 171 and the brim 172, as well as the fastener assembly 174. The fastener assembly 174 has a first fastener 188 located along the inner surface 182 of the prosthetic socket 171 and a second fastener 190 located along an outer surface 183 of the brim 172. The first and second fasteners 188, 190 cooperate to secure one another removably.

The cuff section 173 is arranged to flexibly extend about and upwardly or proximally from a peripheral proximal edge 176 of the prosthetic socket 171. The cuff section 173 has a flexible proximal edge portion 180 extending proximally beyond a peripheral proximal edge 176 of the prosthetic socket 171. The interface section 175 defines a lower or distal portion of the brim extending distally from the cuff section 173.

The interface section 175 includes the second fastener 190, arranged as part of a fastener assembly 174, for removably securing the fastener assembly 174 to the prosthetic socket 171. The interface section 175 has a distal edge 178 extending below the second fastener 190. As shown in FIG. 10, the socket 171 has a band 192 integrated therewith, which carries the first fastener 188. The outer surface 183 of the interface section 175 may include an outer fabric 198 arranged to be attached to the prosthetic socket 171 (e.g., by integrating with the resin during fabrication).

While the fastener assembly in a preferred embodiment shows snap fasteners, other fasteners may be used, including hooks, rings, hook-and-loop, and others.

According to incorporating the socket assembly 170 concepts in a method for fabricating the socket assembly 170, a “receiver” part and a dummy brim may be placed on the residual limb during fabrication. According to the socket assembly 170 concepts, a method for fabricating the socket assembly 170 may include placing a “receiver” part and a brim on the residual limb. The socket is then fabricated as normal. After fabrication, the “receiver” part remains inside the socket; it contains one piece of the fastener (i.e., the first fastener). The arrangement of the socket assembly 170 allows the user to switch between permanent brims, such as brim 172, for different activities, or to compensate for volume changes using the mating part of the snap in the brim.

FIG. 13 shows an embodiment of a brim 200 adapted to a transtibial amputee and corresponding socket. Indeed, the anterior portion 202, the posterior portion 204, the lateral portion 206, and the medial portion 208 may be adapted accordingly. The extent of the cuff relative to the interface section may likewise be adjusted accordingly, wider or narrower, thicker or thinner, depending on the desired flexibility. The brim 200 and its corresponding geometry may be configured according to any of the embodiments above, including those incorporated by reference. The outer surface of the interface brim 200 may include an outer fabric 210 arranged to be attached to the socket 171 (e.g., by integrating with the resin during fabrication).

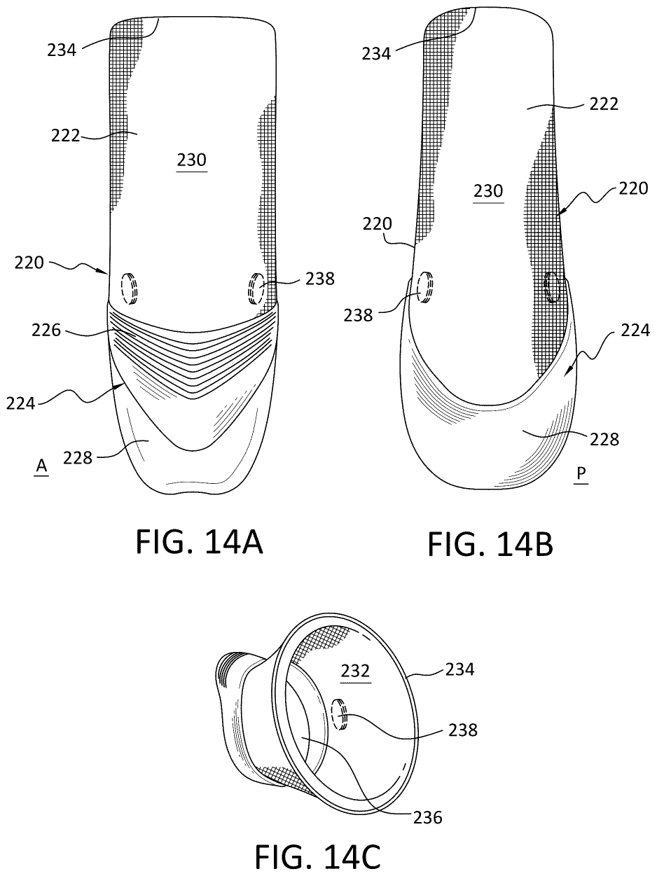

FIGS. 14A-14C illustrate an embodiment of a sleeve 220 having a sleeve portion 222 and a brim 224 located at a distal end of the sleeve portion 222. An exemplary sleeve and discussion of the background thereof is described in U.S. Pat. No. 6,592,539, granted on Jul. 15, 2002, and incorporated herein by reference. The sleeve 220 defines a proximal open end 234 and a distal open end 236 adapted to secure to a socket, as in any of the embodiments mentioned herein.

As shown in FIGS. 14A and 14B, with FIG. 14A illustrating the anterior aspect A and FIG. 14B illustrating the posterior aspect B; the anterior aspect 226 of the brim 224 is contoured differently than the posterior aspect 228 of the brim 224 to accommodate conventional socket shapes.

As shown in FIGS. 14A-14C, the outer surface 230 of the sleeve portion comprises at least one elasticized fabric element. At least the inner surface 232 of the sleeve portion 222 comprises a fabric element coated with a discrete layer of silicone elastomer, which is firmly bonded to the fabric material constituting the sleeve. The silicone elastomer layer is thick and soft, providing a very comfortable fit and an airtight seal between the sleeve and the user's skin. Of note, while it is preferable that the inner surface be entirely coated with an elastomeric material, such as silicone, variations may include spaced-apart sections of the inner surface coated with an elastomeric material or with other suitable materials adapted to achieve the above-stated purpose. Indeed, the inner surface 232 may likewise have sections of different materials and/or properties, adapted to be comfortable for the user and to provide an airtight seal.

The sleeve 220 may be arranged modularly so that it may be attached to a socket, inclusive of the brim, according to any of the means described herein. For exemplary purposes, FIGS. 14A-14B illustrate at least one attachment element 238, such as a snap, hook-and-loop, or other suitable means, for securing to a corresponding attachment element of the socket.

It is understood that not all objects or advantages may be achieved under an embodiment of the disclosure. Those skilled in the art will recognize that a brim may be embodied or conducted, so it achieves or optimizes one advantage or group of advantages as taught herein without achieving other objects or advantages as taught or suggested herein.

The skilled artisan will recognize the interchangeability of various disclosed features. Besides the variations described, other known equivalents for each feature can be mixed and matched by one of ordinary skill in this art to build and use a prosthetic socket under the principles of the present disclosure. Furthermore, the skilled artisan will understand that the features described may be adapted to other prosthetic devices and methods.

Although this disclosure describes certain exemplary embodiments and examples of a brim for a prosthetic socket, it will be understood by those skilled in the art that the present disclosure extends beyond the specifically disclosed components to other alternative embodiments and/or uses of the disclosure and obvious modifications and equivalents thereof. It is intended that the present disclosure should not be limited by the disclosed embodiments described above and may be extended to other applications that may employ the features described.

1. A brim for securing to a proximal end of a prosthetic socket, the socket defining an opening at the proximal end, an outer surface, and an inner surface; the brim being coaxial to a central axis of the socket; the brim comprising:
a cuff section arranged to flexibly extend about and upwardly or proximally from a peripheral proximal edge of the socket; and
an interface section defining a lower or distal portion of the brim and extending distally from the cuff section, the interface section forming a pocket having at least one lip arranged to extend over a proximal end portion of the socket and to secure therewith.

2. The brim of claim 1, wherein the pocket comprises a first lip extending over an exterior side of the proximal end portion and a second lip extending over an interior side of the proximal end portion.

3. The brim of claim 1, wherein the cuff section and interface section are integrally formed of a monolithic elastomeric material.

4. The brim of claim 1, wherein a distal edge of the brim varies in distance from the peripheral proximal edge of the socket about the opening at the proximal end.

5. The brim of claim 1, wherein a proximal edge of the brim varies in height from the peripheral proximal edge of the socket about the opening at the proximal end.

6. The brim of claim 1, wherein an anterior portion of the cuff section has a greater height than a medial or lateral portion thereof.

7. The brim of claim 6, wherein a posterior portion of the cuff section has a greater height than the anterior portion.

8. The brim of claim 7, wherein the posterior portion of the cuff section defines at least one strap arranged laterally relative to the central axis of the socket to form a flexion brim.

9. The brim of claim 8, wherein the at least one strap is confined within a frame section extending outwardly relative to the peripheral proximal edge of the socket.

10. The brim of claim 8, wherein the at least one strap is flexible in a proximal direction and more rigid in a lateral direction.

11. The brim of claim 1, wherein the interface section includes a textile reinforcement layer disposed adjacent the lip to enhance adhesion with a resin of the socket.

12. The brim of claim 11, wherein the reinforcement layer comprises a fibrous material stitched between one or more textile layers.

13. The brim of claim 1, wherein the cuff section is configured to fold downwardly over the outer surface of the socket for donning and doffing.

14. The brim of claim 1, wherein an exterior surface of the interface section includes an outer fabric arranged to secure with resin of the socket.

15. The brim of claim 1, wherein a proximal portion of the brim is attached to a tubular sleeve.

16. A method of fabricating a prosthetic socket having a brim, comprising:
arranging a socket blank formed of fiber braids in a conical formation about a stump model;
positioning a first textile layer over the socket blank;
cutting and tapering the fiber braids to form a tapered portion of the socket blank;
placing a brim having an interface forming a pocket over a proximal end of the socket blank such that a proximal end of the fiber braids is received within the pocket; and
applying a resin to impregnate the textile layer and integrate the brim with the socket.

17. The method of claim 16, wherein the brim includes first and second lips, and the step of placing the brim comprises tucking the fiber braids between the first and second lips prior to resin impregnation.

18. The method of claim 16, further comprising positioning at least one additional textile layer over the socket blank and the first textile layer to cover the tapered portion of the fiber braids.

19. The method of claim 16, further comprising applying a cosmetic textile layer over the socket and brim to provide a uniform exterior surface free of discernible transition lines.

20. A prosthetic socket system comprising:
a prosthetic socket defining a proximal end, an outer surface, and an inner surface;
a brim attached about the proximal end, the brim including a cuff section arranged to flexibly extend proximally from a peripheral proximal edge of the socket and an interface section forming a pocket having at least one lip extending over a proximal end portion of the socket;
at least one textile layer arranged along the socket and attached to the brim;
a reinforcement layer disposed at the interface section of the brim; and
a cured resin impregnating the at least one textile layer and securing the brim, the reinforcement layer, and the socket into a continuous, integrated structure accommodating variation in socket geometry, flexibility, and user anatomy.
